(19) United States
(12) Patent Application Publication
(10) Pub. No.: US 2013/0245372 A1
(43) Pub. Date: Sep. 19, 2013

(54) ENDOTRACHEAL INTUBATION ASSISTANCE APPARATUS

(71) Applicant: GENTLE AND TENDER CO., LTD., Taipei City (TW)

(72) Inventor: Po-Han LO, Taipei City (TW)

(73) Assignee: GENTLE AND TENDER CO., LTD., Taipei City (TW)

(21) Appl. No.: 13/793,039

(22) Filed: Mar. 11, 2013

(30) Foreign Application Priority Data
Mar. 13, 2012 (TW) 101108479

(51) Int. Cl. A61M 16/04; A61B 1/267; A61B 1/04

(52) U.S. Cl. CPC A61M 16/0488; A61B 1/04; A61B 1/267
USPC 600/109; 128/200.26

(57) ABSTRACT

An endotracheal intubation assistance apparatus is adapted to assist in insertion of an endotracheal tube into the trachea of a patient, and includes a movable tubular stylet, a graspable controller, and a viewing device. The stylet has a leading section, a body section, a tail section, and two slits extending through the body section and the tail section. The tail section is divided by the slits into first and second driven sheets. The viewing device includes an elongate body and a viewing head. The elongate body and the viewing head are movable through the graspable controller, and are extendable outwardly from the leading section. When the first and second driven sheets move relative to each other, the leading section swings synchronously a distal end of the endotracheal tube and the viewing head.

### CROSS-REFERENCE TO RELATED APPLICATION

This application claims priority of Taiwanese Application No. 101108479, filed on Mar. 13, 2012.

### BACKGROUND OF THE INVENTION

1. Field of the Invention

This invention relates to a medical instrument, and more particularly to an endotracheal intubation assistance apparatus that has a simple structure, a low cost, and a high clinical applicability.

2. Description of the Related Art

It is important for endotracheal intubation to be performed in a safety and quick manner. Otherwise, teeth or laryngeal tissue of the patient may be hurt, or an endotracheal tube is inserted erroneously into the esophagus disposed behind the trachea, thereby resulting in occurrence of severe complications, such as hypoxia.

Referring to FIG. 1, a conventional endotracheal tube 10 is associated with a standard stylet 20. The endotracheal tube 10 includes a tube body 101 having a predetermined curvature and flexibility, a distal end 102, a proximal end 103, a connector 104 sleeved on the proximal end 103, and an inflatable cuff 105 adjacent to the distal end 102. The standard stylet 20 can be shaped manually to change the curvature of the endotracheal tube 10 (i.e., the angle of the distal end 102). When the angle is not correct, it is necessary to draw an assembly of the endotracheal tube 10 and the standard stylet 20 out of the mouth of the patient for shape adjustment. Upon the completion of the shape adjustment to align the distal end 102 with the vocal cords of the patient, the endotracheal tube 10 is inserted into the trachea.

The standard stylet 20 may be replaced with a bougie having a greater length, or a lightwand, as disclosed in US NO. 20080017195. A leading end of the bougie or the lightwand is inserted into the trachea. Under guide of the bougie or the lightwand, the endotracheal tube can be passed fully through the opening defined by the vocal cords. However, the medical personnel must have the related experience and skill. Furthermore, such an endotracheal intubation device may not be suitable for patients more difficult to intubate.

U.S. Pat. Nos. 3,802,440, 4,949,716, 5,259,377, and 5,791,338 disclose various auxiliary devices for adjusting the curvature of the endotracheal tube, each of which includes a movable stylet and a control mechanism for activating the movable stylet in a manner more efficient than the above-mentioned manual shaping manner.

To perform the above intubation procedures, a blade of a laryngoscope (e.g., disclosed in U.S. Pat. No. 3,638,644) is required to press against the tongue of the patient for allowing light to be emitted into the throat of the patient. However, if the mouth of the patient cannot open to a larger extent, it is difficult to put the laryngoscope into the mouth. Or, although the laryngoscope can be inserted into the mouth, the throat of the patient cannot be seen clearly by the medical personnel. In this case, blind intubation may be tried.

U.S. Pat. Nos. 3,669,098, 5,327,881, 6,319,195, 6,539,942, 7,458,375, and US Pub. No. 2008/0236575 disclose a viewing device that is built in a movable stylet. The viewing device is configured as an optical fiber assembly or a small video camera, and is used with an ocular or display for showing images. This can eliminate the use of a bulky laryngoscope to reduce anatomical distortion, and can perform real time observation of the vocal cords. In this manner, leading ends of the movable stylet and the endotracheal tube can be moved into the proximity of the vocal cords, followed by inserting only the endotracheal tube into the trachea. However, in a situation where the opening defined by the vocal cords is small, when anatomical distortion or the throat swelling occurs, or when the cough reflex is triggered, the resistance makes the movable stylet deformed and difficult to manipulate. Furthermore, if the angle formed between the longitudinal directions of the distal end of endotracheal tube and the trachea is too large, it is also difficult to intubate.

Since the movable stylet typically has a complex structure, and cooperates with the viewing device to form one piece, such a one piece structure would not be designed to be disposable.

To overcome the blind condition of the bougie or the lightwand, U.S. Pat. No. 6,978,784 and US Pub. No. 2007/0175482 disclose a viewing device that permits an endotracheal tube to be sleeved thereon and that can serve as a guide device. The viewing device is first inserted into the trachea. Subsequently, the endotracheal tube is also inserted into the trachea along the viewing device. For awake and obedient patients, such an endotracheal intubation process has been considered to be a current gold standard. However, the stiffness and maneuverability of the viewing device are not sufficient, so that the skill standard of the medical personnel for performing such endotracheal intubation process is relatively high. U.S. Pat. No. 6,257,236 discloses a bronchoscope for facilitating insertion of a stylet into the trachea to allow an endotracheal tube to be sleeved on the stylet and then pushed into the trachea. However, the stiffness and maneuverability of the bronchoscope are also not sufficient. U.S. Pat. No. 6,508,757 discloses a malleable material that is sleeved on a viewing device to increase the stiffness of the viewing device. However, for angle adjustment, removing an assembly of the viewing device and the malleable material from the mouth may be needed, as required by the standard stylet. U.S. Pat. No. 6,146,402 discloses a guide tube introducer for facilitating a guide wire to be placed into the trachea. However, the guide tube introducer is not malleable and flexible, and cannot be preloaded with an endotracheal tube due to the fact that the guide tube introducer must be removed prior to mounting the endotracheal tube.

### SUMMARY OF THE INVENTION

The object of this invention is to provide an endotracheal intubation assistance apparatus that has a simple structure, a low cost, and a high clinical applicability.

According to this invention, an endotracheal intubation assistance apparatus is adapted to assist in insertion of an endotracheal tube into the trachea of a patient, and includes a movable tubular stylet, a graspable controller, and a viewing device. The stylet has a leading section, a body section, a tail section, and two slits extending through the body section and the tail section. The tail section is divided by the slits into first and second driven sheets. The viewing device includes an elongate body and a viewing head. The elongate body and the viewing head are movable through the graspable controller, and are extendable outwardly from the leading section. When the first and second driven sheets move relative to each other, the leading section swing synchronously a distal end of the endotracheal tube and the viewing head.

By controlling relative movement between the first and second driven sheets, the leading section can swing the distal end of the endotracheal tube and the viewing head synchronously therewith, so as to allow the elongate body to move toward the trachea by a predetermined distance for guiding the endotracheal tube to move toward the trachea along the elongate body. In this manner, the success rate of endotracheal intubation can be promoted effectively, and intubation time period can be reduced.

### BRIEF DESCRIPTION OF THE DRAWINGS

These and other features and advantages of this invention will become apparent in the following detailed description of a preferred embodiment of this invention, with reference to the accompanying drawings, in which:

FIG. 1 is a schematic side view of a conventional assembly of an endotracheal tube and a standard stylet;

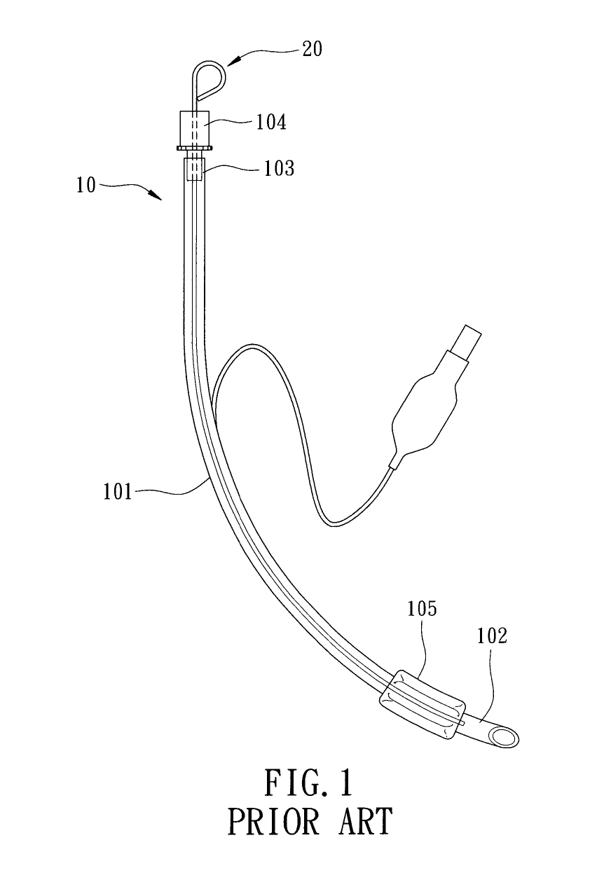

FIG. 2 is a schematic view of the preferred embodiment of an endotracheal intubation assistance apparatus according to this invention, illustrating that a leading section of a movable tubular stylet and a distal end of an endotracheal tube are located adjacent to the vocal cords;

FIG. 3 is a perspective view of the movable tubular stylet of the preferred embodiment;

FIG. 4 is a sectional view of the movable tubular stylet of the preferred embodiment;

FIG. 5 is a fragmentary perspective view of the preferred embodiment, illustrating a graspable controller, the movable tubular stylet, and a viewing device;

FIG. 6 is a sectional view of the preferred embodiment;

FIG. 7 is a fragmentary sectional view of the preferred embodiment, illustrating connection between a driving device of the graspable controller and the movable tubular stylet;

FIG. 8 is a fragmentary exploded perspective view of the preferred embodiment;

FIG. 9 is a partially sectional view of the preferred embodiment, illustrating a retaining position of a retaining plate of the graspable controller;

FIG. 10 is a fragmentary perspective view of the preferred embodiment, illustrating an elongate body and a viewing head of the viewing device;

FIG. 11 is a schematic view of the preferred embodiment, illustrating operation of an operating member of the graspable controller and the movable tubular stylet;

FIG. 12 is view similar to FIG. 9 but illustrating a release position of the retaining plate;

FIG. 13 is a schematic view of the preferred embodiment, illustrating that the elongate body of the viewing device is inserted into the trachea;

FIG. 14 is a schematic view of the preferred embodiment, illustrating that the endotracheal tube and the movable tubular stylet are inserted into the trachea along the elongate body; and

FIG. 15 is a schematic view of the preferred embodiment, illustrating that the movable tubular stylet and the viewing device are removed, and only the endotracheal tube is left in the trachea.

### DETAILED DESCRIPTION OF THE PREFERRED EMBODIMENT

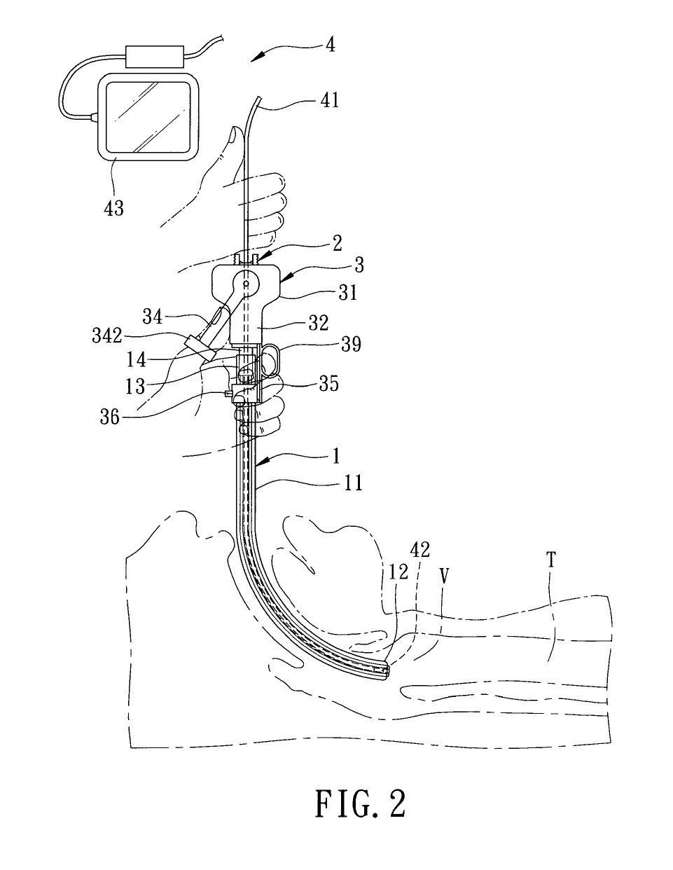

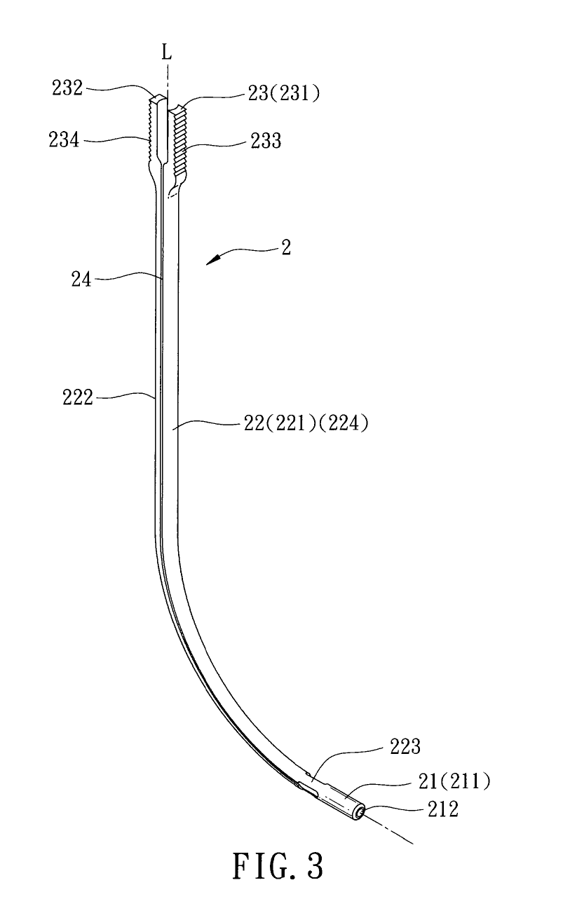

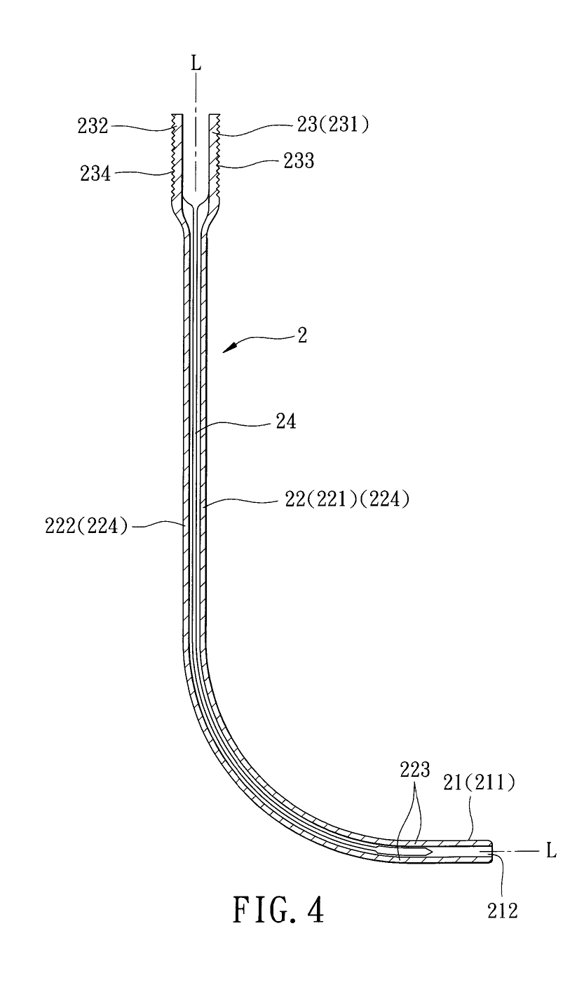

Referring to FIGS. 2, 3, and 4, the first preferred embodiment of an endotracheal intubation assistance apparatus according to this invention is adapted to assist in insertion of an endotracheal tube 1 into the trachea (T) of a patient. The endotracheal tube 1 includes a tube body 11 having a predetermined curvature and flexibility, a distal end 12, a proximal end 13, and a connector 14 sleeved on the proximal end 13. Alternatively, the connector 14 may be omitted from the endotracheal tube 1. The endotracheal intubation assistance apparatus includes a movable tubular stylet 2, a graspable controller 3, and a viewing device 4.

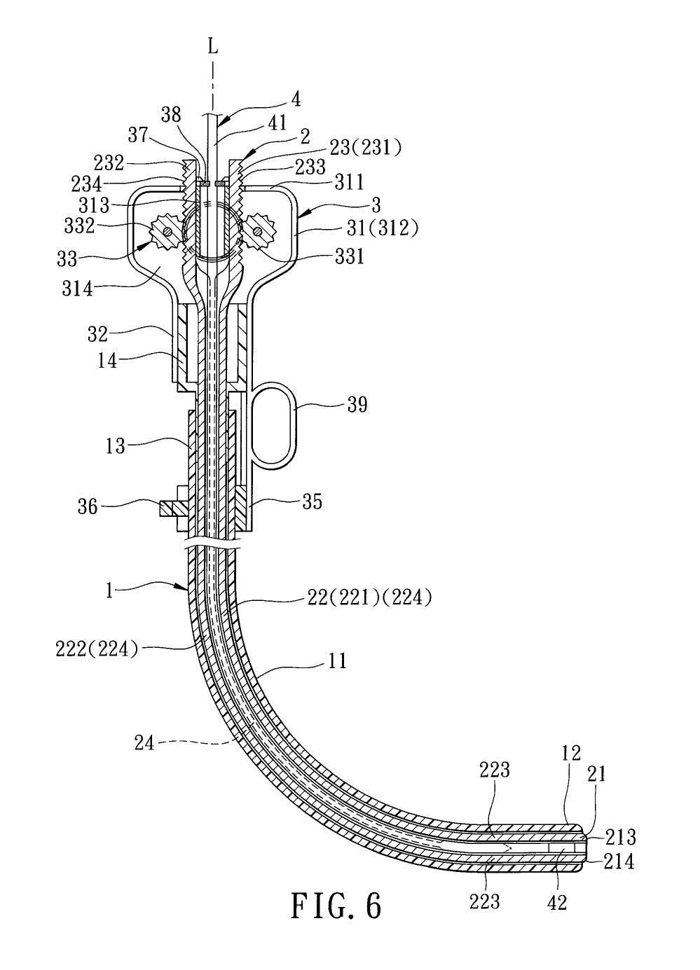

The movable tubular stylet 2 is flexible, and has a leading section 21, a tail section 23, a body section 22 connected between the leading section 21 and the tail section 23 along the length (L) of the movable tubular stylet 2, and two slits 24 extending through the body section 22 and the tail section 23. The movable tubular stylet 2 is formed from a plastic material (such as Teflon) as one piece. The leading section 21 and the body section 22 are adapted to extend in the tube body 11 of the endotracheal tube 1. With additional reference to FIG. 6, the leading section 21 is configured as a cylinder 211 having an inner bore 212, and a leading end 213 extending outwardly from the distal end 12 of the endotracheal tube 1 and having a chamfered outer periphery 214.

The body section 22 is divided by the slits 24 into a first strip 221 and a second strip 222. Each of the first and second strips 221, 222 has a weakened area 223 adjacent to the leading section 21, and an action area 224 disposed between the weakened area 223 and the tail section 23 and having a cross sectional area greater than that of the weakened area 223.

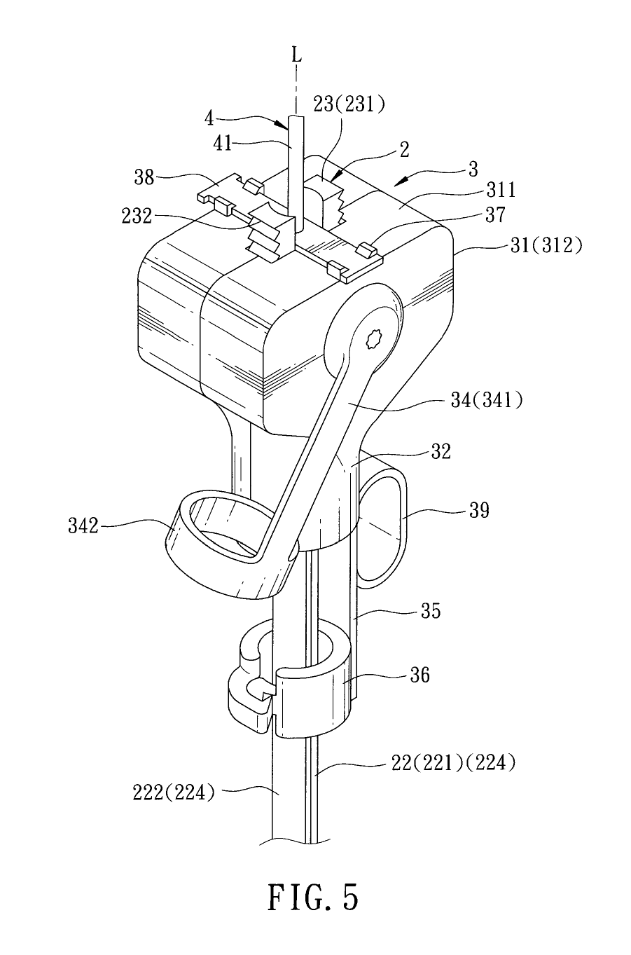

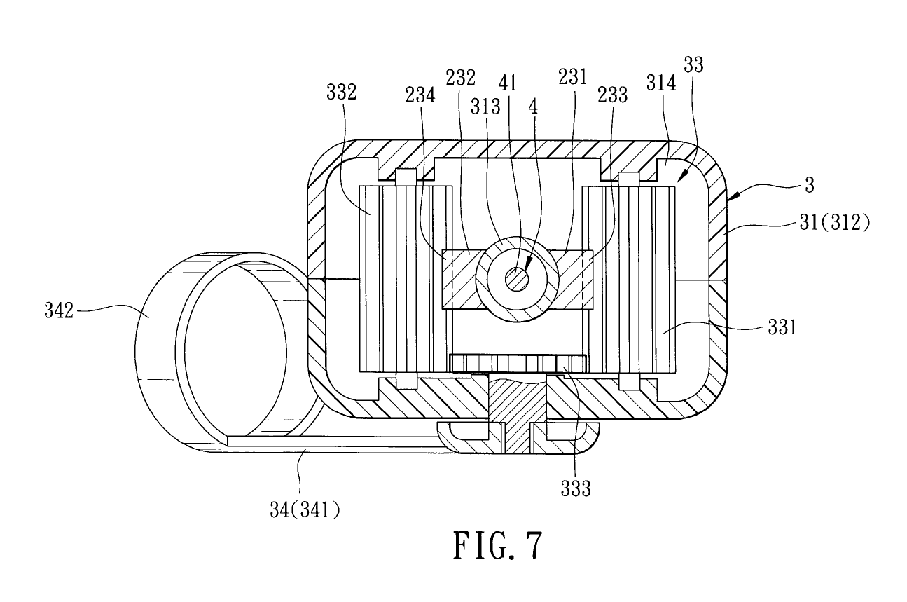

The tail section 23 is divided by the slits 24 into a first driven sheet 231 connected to the first strip 221, and a second driven sheet 232 connected to the second strip 222. The first and second driven sheets 231, 232 are provided respectively with first and second racks 233, 234 extending along the length (L) of the movable tubular stylet 2. In this embodiment, the first and second driven sheets 231, 232 of the tail section 23 are formed respectively and integrally with the first and second strips 221, 222 of the body section 22. Alternatively, the first and second driven sheets 231, 232 are made of a rigid material different from that of the first and second strips 221, 222, such as metal, and are connected to the first and second strips 221, 222, respectively. With further reference to FIGS. 5 and 7, the graspable controller 3 includes a main body 31, a sleeve 32 connected to and disposed under the main body 31, a driving mechanism 33 disposed in the main body 31, an operating member 34 operable for activating the driving mechanism 33, an extension grip 35 connected to and extending downwardly from the sleeve 32, a retaining clamp 36 connected to the extension grip 35, a rail 37 disposed on the main body 31, a retaining plate 38 disposed movably on the rail 37 and permitting the viewing device 4 to extend therethrough, and a tubular finger-retaining member 39 connected to a side surface of the extension grip 35 facing away from the retaining clamp 36.

The main body 31 includes a top wall 311, a surrounding wall 312 connected between the top wall 311 and the sleeve 32, and a guide tube 313 extending downwardly from the top wall 311 and aligned with the sleeve 32.

The surrounding wall 312 defines a space 314 in spatial communication with the sleeve 32. The guide tube 313 permits the viewing device 4 to extend therethrough.

The sleeve 32 is adapted to be sleeved on the connector 14, and permits the tail section 23 of the movable tubular stylet 2 to extend therethrough. If the connector 14 is omitted from the endotracheal tube 1, the sleeve 32 can be sleeved directly on the proximal end 13 of the endotracheal tube 1.

The driving mechanism 33 is disposed in the space 314, and includes a first driving member 331 connected to the first driven sheet 231, a second driving member 332 connected to the second driven sheet 232, and a common gear 333 disposed pivotally on the main body 31. In this embodiment, each of the first and second driving members 331, 332 is configured as a pinion. The guide tube 313 extends between the first and second driving members 331, 332, and has an outer surface in contact with the first and second driven sheets 231, 232, so as to maintain the first and second driven sheets 231, 232 to mesh with the first and second driving members 331, 332.

The first and second driving members 331, 332 are disposed pivotally on the main body 31, and are located respectively at two sides of the common gear 333. The first driving member 331 meshes with the common gear 333 and the first rack 233. The second driving member 332 meshes with the common gear 333 and the second rack 234. The first and second driving members 331, 332 can be driven to move synchronously the first and second driven sheets 231, 232 in opposite directions along the length (L) of the movable tubular stylet 2.

The operating member 34 is disposed on and outwardly of the surrounding wall 312, is connected to the common gear 333, and is operable for driving rotation of the common gear 333 in two opposite directions. The operating member 34 includes a connecting rod 341, and a finger ring 342 disposed on an end of the connecting rod 341. An opposite end of the connecting rod 341 is connected to the common gear 333. The extension grip 35 and the finger ring 342 of the operating member 34 are located respectively at two sides of the sleeve 32, and are disposed respectively under the first and second driving members 331, 332. The retaining clamp 36 is disposed on the extension grip 35, and is located directly under the sleeve 32 for clamping releasably the tube body 11 of the endotracheal tube 1.

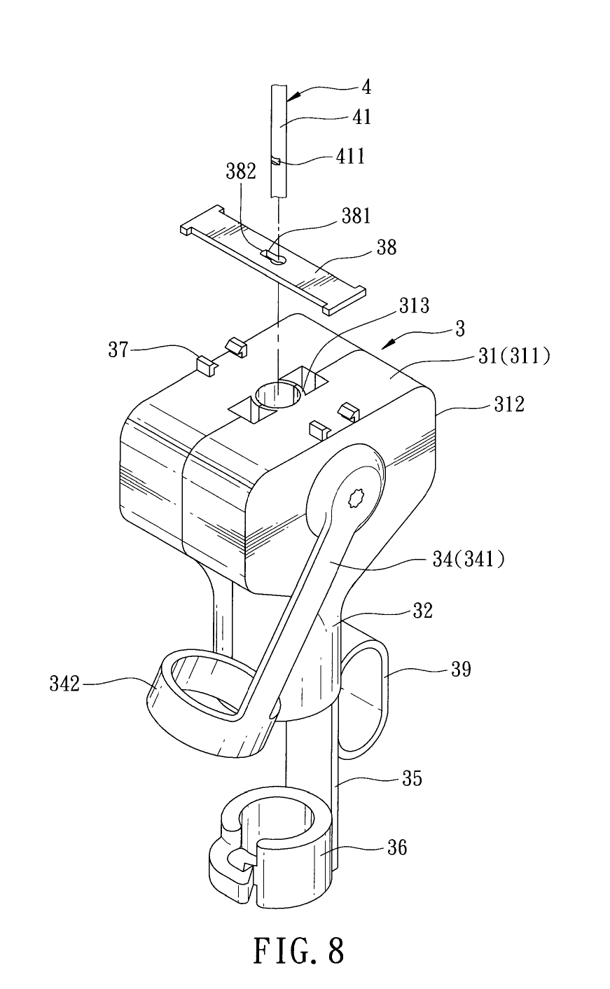

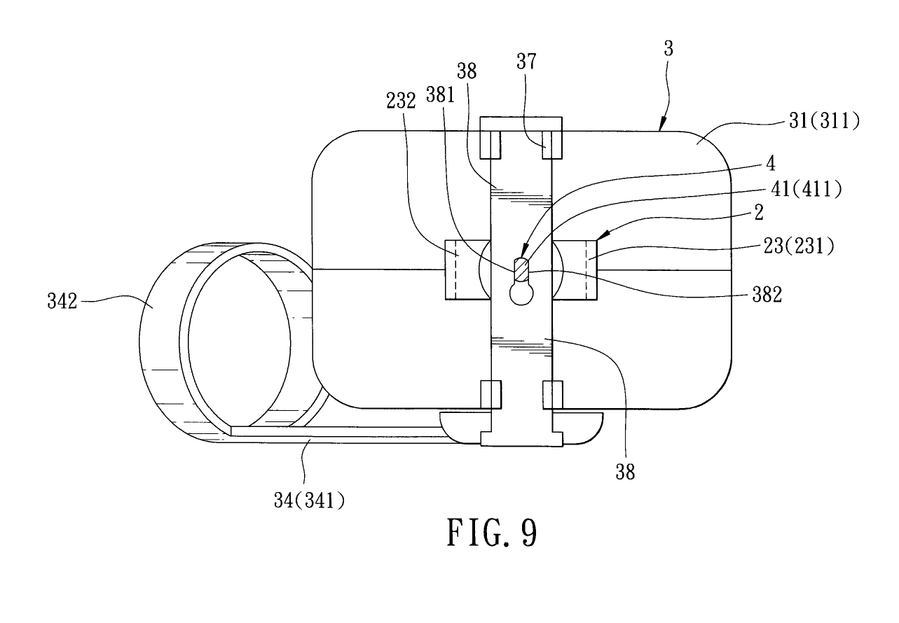

With further reference to FIGS. 8 and 9, the rail 37 is disposed on the top wall 311 of the main body 31. The retaining plate 38 is formed with a hole 381 that has a retaining hole portion 382.

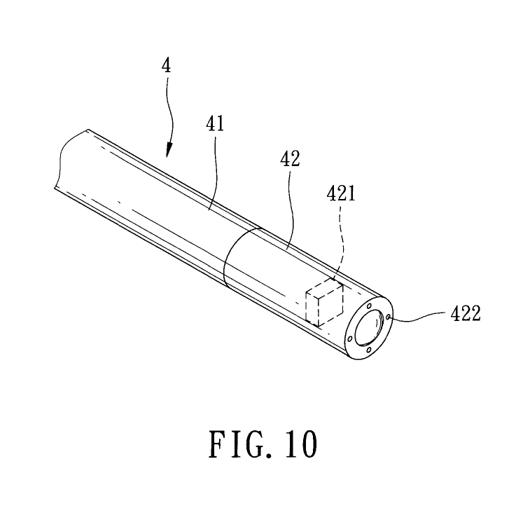

With particular reference to FIGS. 2, 8, and 10, the viewing device 4 includes a flexible elongate body 41 having a diameter of about 4 mm, a viewing head 42 disposed on a leading end of the elongate body 41 and having a length of about 10 mm, and a display 43 electrically connected to the elongate body 41. The elongate body 41 extends through the hole 381, and has a contracted portion 411 engageable with the retaining hole portion 382. The contacted portion 411 has a cross sectional area smaller than that of the remaining portion of the elongate body 41. The viewing head 42 includes a small video camera 421 disposed therein, and at least one light-emitting member 422 disposed thereon. Alternatively, the viewing device 4 may be an optical-fiber viewing device.

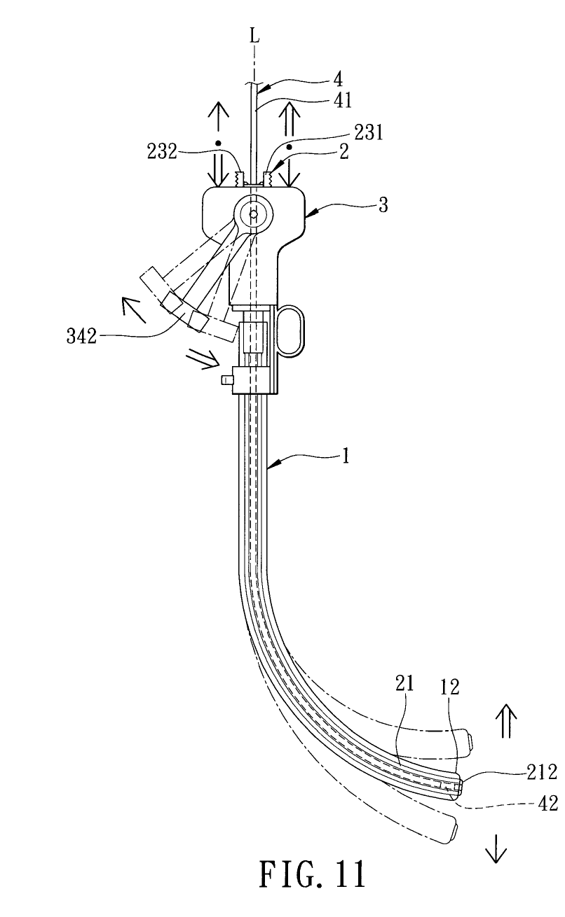

With particular reference to FIGS. 2, 6, and 11, the elongate body 41 and the viewing head 42 can be moved in the graspable controller 3 along the length (L) of the movable tubular stylet 2, and can extend outwardly from the inner bore 212 in the leading section 21. When the finger ring 342 is pivoted downwardly, the first and second driven sheets 231, 232 are moved in opposite directions along the length (L) of the movable tubular stylet 2. Hence, forces are transmitted to the weakened areas 223 of the first and second strips 221, 222 through the action areas 224 of the first and second strips 221, 222, so that the distal end 12 of the endotracheal tube 1 and the viewing head 42 are swung upwardly. When the finger ring 342 is pivoted upwardly, the first and second driven sheets 231, 232 are moved in opposite directions along the length (L) of the movable tubular stylet 2. Hence, forces are transmitted to the weakened areas 223 of the first and second strips 221, 222 through the action areas 224 of the first and second strips 221, 222, so that the distal end 12 of the endotracheal tube 1 and the viewing head 42 are swung downwardly. As such, the cylinder 211 of the leading section 21 is sufficiently stiff to support the viewing device 4 and the endotracheal tube 1, and the weakened areas 223 have an increased flexibility so as to allow the leading section 21 to swing easily.

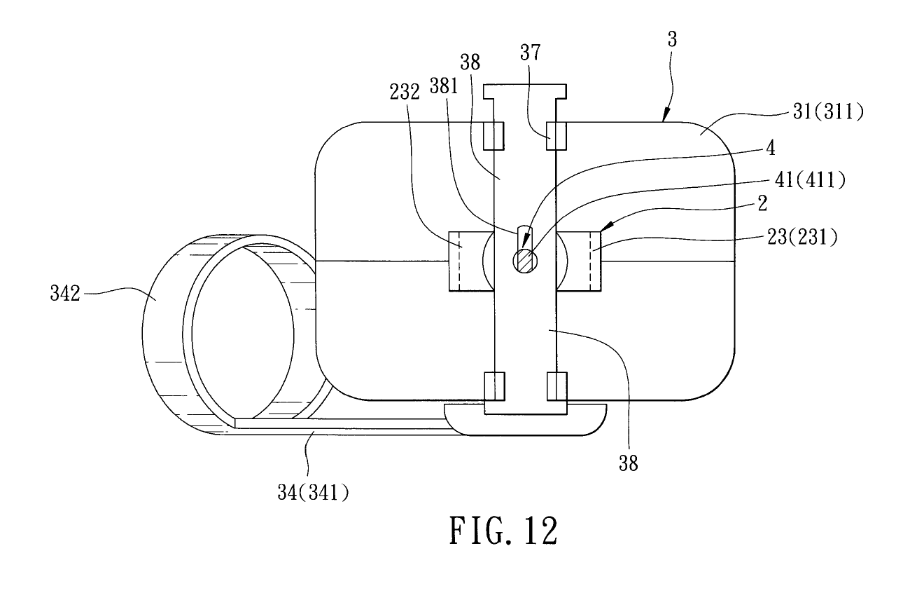

With particular reference to FIGS. 5, 6, 9, and 12, during use, the retaining plate 38 is movable relative to the elongate body 41 between a release position shown in FIG. 12, and a retaining position shown in FIG. 9. At the release position, the contracted portion 411 is removed from the retaining hole portion 382 so as to allow for movement of the elongate body 41 relative to the retaining plate 38. At the retaining position, the contracted portion 411 is engaged within the retaining hole portion 382 so as to prevent movement of the elongate body 41 relative to the retaining plate 38. In this position, the viewing head 42 is received wholly in the leading section 21 for avoiding accession of the dribble to the viewing head 42.

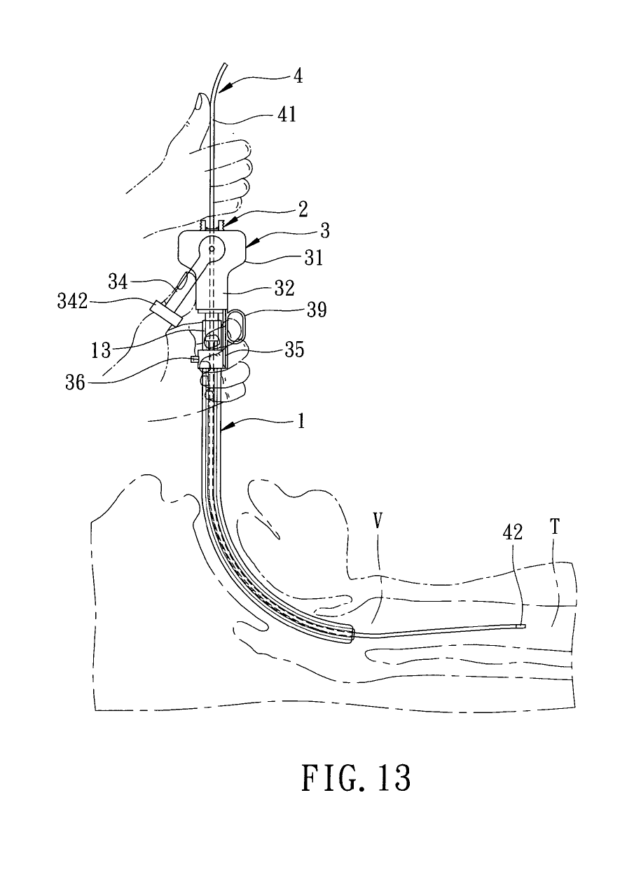

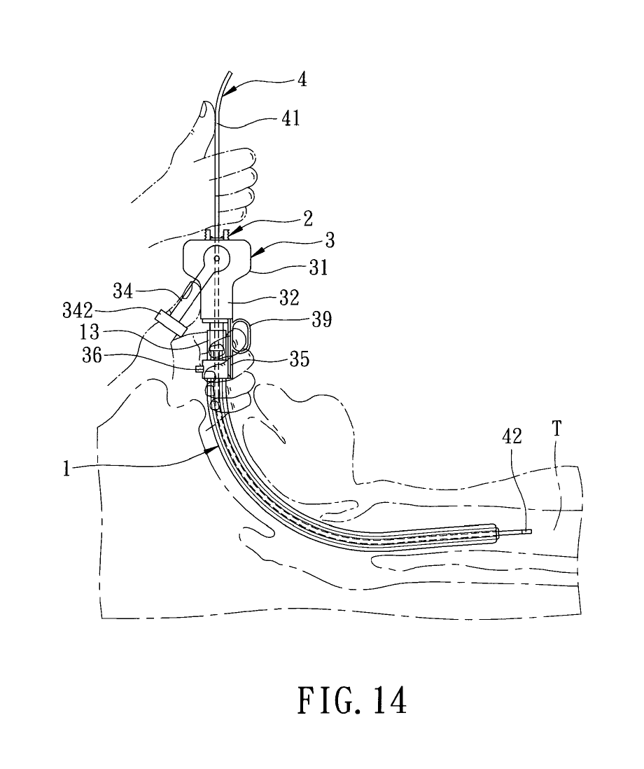

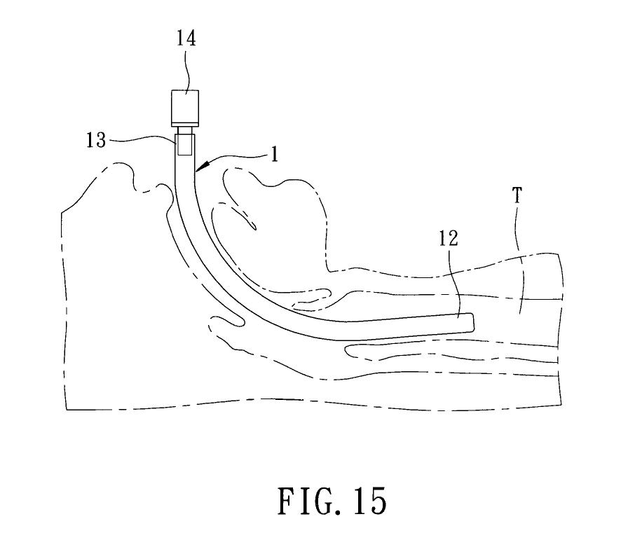

With particular reference to FIGS. 2 and 9, during an endotracheal intubation operation, the retaining plate 38 is first placed at the retaining position. Next, the user holds the graspable controller 3 with one hand in such a manner that, the thumb is inserted into the finger ring 342, the index finger is inserted into the finger-retaining member 39, and the remaining three fingers grips the extension grip 35 of the graspable controller 3 and the proximal end 13 of the endotracheal tube 1. Afterwards, the operating member 34 is operated with the thumb to control the movable tubular stylet 2 to thereby swing the distal end 12 of the endotracheal tube 1 and the viewing head 42 of the viewing device 4 upwardly or downwardly. As soon as the display 43 shows that the viewing head 42 is aligned with the opening defined by the vocal cords (V), the retaining plate 38 is moved to the release position, and the elongate body 41 is pushed with the other hand to move toward the trachea (T) by a predetermined distance, as shown in FIG. 13. At this time, the endotracheal tube 1 and the movable tubular stylet 2 are pushed into the trachea (T) along the elongate body 41, as shown in FIG. 14. Finally, the retaining clamp 36 is released, and the movable tubular stylet 2 and the elongate body 41 are drawn out of the endotracheal tube 1 so that the endotracheal tube 1 is left in the trachea (T), thereby completing the endotracheal intubation operation, as shown in FIG. 15.

To sum up, the endotracheal intubation assistance apparatus of this invention has the following advantages:
1. Different from the above-mentioned conventional assembly of a movable stylet and a viewing device that cannot move relative to each other, according to this invention, the viewing device 4 can be moved through the graspable controller 3 and the movable tubular stylet 2, such that both allocation of the vocal cords (V) at the first stage and establishment of a guide into the trachea (T) at the second stage can be achieved with relative ease, thereby promoting effectively the success rate of endotracheal intubation and reducing intubation time period.
2. Since the movable tubular stylet 2 is formed mainly from the plastic material as one piece, and is not formed integrally with the viewing device 4, an assembly of the movable tubular stylet 2 and the viewing device 4 is made at a low cost, is easy to maintain, repair, and sterilize, and can be designed to be disposable to eliminate the sterilization procedure and cost.
3. The graspable controller 3 is convenient to operate. This can reduce the training time, and can promote clinical applicability.
4. Through the design of the extension grip 35, the medical personnel can hold firmly the extension grip 35 and the proximal end 13 of the endotracheal tube 1 with one hand. As such, sway of the endotracheal tube 1 and the viewing device 4 would be reduced to improve the reliability of the endotracheal tube 1 and the viewing device 4.

it should be noted that, in this embodiment, the endotracheal tube 1 is inserted into the trachea (T) through the mouth. In alternative embodiments, the endotracheal tube 1 can be inserted into the trachea (T) through the nostril.

With this invention thus explained, it is apparent that numerous modifications and variations can be made without departing from the scope and spirit of this invention. It is therefore intended that this invention be limited only as indicated by the appended claims.

I claim:

1. An endotracheal intubation assistance apparatus adapted for assisting in insertion of an endotracheal tube into the trachea of a patient, the endotracheal tube including a flexible tube body having a predetermined curvature, a distal end, and a proximal end, said endotracheal intubation assistance apparatus comprising:
a movable tubular stylet being flexible and having a leading section, a tail section, a body section connected between said leading section and said tail section along length of said movable tubular stylet, and two slits extending through said body section and said tail section, said leading section being configured as a cylinder and having an inner bore, said body section being divided by said slits into a first strip and a second strip, said tail section being divided by said slits into a first driven sheet connected to said first strip, and a second driven sheet connected to said second strip, said leading section and said body section being adapted to extend in the endotracheal tube;
a graspable controller including a main body, a sleeve connected to and disposed under said main body, a driving mechanism disposed on said main body, and an operating member operable for activating said driving mechanism, said sleeve being adapted to be sleeved on the proximal end of the endotracheal tube and permitting said tail section of said movable tubular stylet to extend therethrough, said driving mechanism including a first driving member connected to said first driven sheet, and a second driving member connected to said second driven sheet; and
a viewing device including a flexible elongate body and a viewing head disposed on a leading end of said elongate body, said elongate body and said viewing head being movable in said graspable controller and being extendable outwardly from the inner bore in the movable tubular stylet such that, when said first and second driven sheets are driven to move relative to each other along the length of said movable tubular stylet, said leading section can swing the distal end of the endotracheal tube and said viewing head synchronously therewith.

2. The endotracheal intubation assistance apparatus as claimed in claim 1, wherein said movable tubular stylet is formed as one piece.

3. The endotracheal intubation assistance apparatus as claimed in claim 2, wherein each of said first and second strips of said body section of said movable tubular stylet has a weakened area adjacent to said leading section, and an action area disposed between said weakened area and said tail section and having a cross sectional area greater than said weakened area.

4. The endotracheal intubation assistance apparatus as claimed in claim 2, wherein said leading section of said movable tubular stylet further has a leading end extending outwardly from the distal end of the endotracheal tube and having a chamfered outer periphery.

5. The endotracheal intubation assistance apparatus as claimed in claim 1, wherein said first and second driven sheets of said movable tubular stylet are provided respectively with first and second racks extending along the length of said stylet, said driving mechanism of said graspable controller further including a common gear disposed on said main body, said first and second driving members being disposed pivotally on said main body and located respectively at two sides of said common gear, said first driving member being a pinion meshing with said common gear and said first rack, said second driving member being a pinion meshing with said common gear and said second rack, said first and second driving members being driven to move synchronously said first and second driven sheets in opposite directions along the length of said movable tubular stylet, said operating member being connected to said common gear for driving rotation of said common gear in two opposite directions.

6. The endotracheal intubation assistance apparatus as claimed in claim 5, wherein said main body of said graspable controller includes a top wall, a surrounding wall connected between said top wall and said sleeve, and a guide tube extending downwardly from said top wall, said surrounding wall defining a space in spatial communication with said sleeve, said driving mechanism being disposed in said space, said guide tube being aligned with said sleeve and extending through said first and second driving members, said guide tube permitting said viewing device to extend therethrough and having an outer surface in contact with said first and second driven sheets, so as to maintain said first and second driven sheets to mesh with said first and second driving members, respectively.

7. The endotracheal intubation assistance apparatus as claimed in claim 6, wherein said operating member is disposed on and outwardly of said surrounding wall, and includes a connecting rod, and a finger ring disposed on an end of said connecting rod, an opposite end of said connecting rod being connected to said common gear.

8. The endotracheal intubation assistance apparatus as claimed in claim 7, wherein said graspable controller further includes an extension grip connected to and extending downwardly from said sleeve, said extension grip and said finger ring of said operating member being located respectively at two sides of said sleeve and disposed respectively under said first and second driving members.

9. The endotracheal intubation assistance apparatus as claimed in claim 8, wherein said graspable controller further includes a retaining clamp connected to said extension grip and adapted for clamping releasably the tube body of the endotracheal tube.

10. The endotracheal intubation assistance apparatus as claimed in claim 1, wherein said viewing head of said viewing device is provided with a small video camera disposed therein, and at least one light-emitting member disposed thereon.

11. The endotracheal intubation assistance apparatus as claimed in claim 1, wherein said graspable controller further includes a rail disposed on said main body, and a retaining plate disposed movably on said rail and formed with a hole permitting extension of said elongate body of said viewing device therethrough, said hole in said retaining plate having a retaining hole portion, said elongate body having a contracted engagement portion received within said retaining hole, said contracted portion having a cross sectional area smaller than that of the remaining portion of said elongate body, said retaining plate being movable relative to said elongate body between a release position and a retaining position such that, when said retaining plate is at the release position, said contracted portion is removed from said retaining hole portion so as to allow for movement of said elongate body relative to said retaining plate, and when said retaining plate is at the retaining position, said contracted portion is engaged within said retaining hole portion so as to prevent movement of said elongate body relative to said retaining plate.

12. The endotracheal intubation assistance apparatus as claimed in claim 11, wherein, when said retaining plate is at the retaining position, said viewing head is received wholly within said leading section.
